(19) United States
(12) Patent Application Publication
(10) Pub. No.: US 2023/0404937 A1
(43) Pub. Date: Dec. 21, 2023

(54) NOVEL DISINTEGRATION ORAL FILM FORMULATION WITH A CONTROLLED OR SUSTAINED ACTIVE RELEASE

(71) Applicant: INTELGENX CORP., St-Laurent (CA)

(72) Inventors: Billal TIR, Montréal (CA); Carolin MADWAR, Montréal (CA); Nadine PAIEMENT, St-Laurent (CA)

(73) Assignee: INTELGENX CORP., St-Laurent, QC (CA)

(21) Appl. No.: 18/208,177

(22) Filed: Jun. 9, 2023

Related U.S. Application Data
(60) Provisional application No. 63/350,823, filed on Jun. 9, 2022.

(51) Int. Cl. A61K 9/70; A61K 9/00; A61K 31/00; A61K 31/439; A61K 47/38; A61K 47/34; A61K 47/26

(52) U.S. Cl. CPC A61K 9/7007; A61K 9/006; A61K 31/658; A61K 47/26; A61K 47/38; A61K 47/34; A61K 31/439

(57) ABSTRACT

Oral film formulations comprising at least one hydrophobic active agent; and a polymeric matrix at least partly composed of hydrophobic or insoluble excipients, or a combination thereof, to facilitate a controlled or sustained dissolution. Wherein when the oral film formulation has an unfavorable environment for fast dissolution, it exhibits partial disintegration within 1 to 10 minutes and a sustained rate of dissolution as confirmed by a disintegration test conducted in a limited volume petri dish, and wherein the film undergoes disintegration into smaller and smaller pieces.

### CROSS-REFERENCE TO RELATED APPLICATIONS

The present application claims priority to U.S. application No. 63/350,823 filed on Jun. 9, 2022. This document is hereby incorporated by reference in its entirety.

### FIELD OF THE DISCLOSURE

This disclosure relates to oral film formulations and processes for preparing oral film dosage forms, and more particularly to the preparation of oral film formulations that have a controlled rate of dissolution employing disintegration. This disclosure also relates to oral film formulations and processes that improve oral absorption of complex active agents.

### BACKGROUND OF THE DISCLOSURE

It is often desirable to administer a pharmaceutical ingredient using an oral film dosage form. Oral film dosage forms have several advantages when compared with tablet and capsules. Many people have difficulty swallowing tablets and capsules, and risk choking while attempting to swallow solid oral dosage forms, but can self-administer a film dosage form without difficulty.

Oral films refer to a type of dosage form that is distinctly different from pills, tablets, caplets, and capsules, and in which the dosage form is a thin strip of material. It will be understood that the terms “oral film” and “oral film formulation” include delivery systems of any thickness, including films, film strips, discs, wafers, sheets, and the like, in any shape.

An oral film is a drug delivery system incorporating biologically active ingredient(s) in a polymeric matrix. Upon placement of the film in the oral cavity and hydration of the film by saliva, the biologically active ingredient(s) incorporated within the film are released from the film matrix, via diffusion and/or due to the erosion/disintegration or dissolution of the film matrix. The bioactive material released from the film matrix is sometimes cleared from the oral cavity by swallowing, but preferably, the bioactive material will be absorbed through the mucous membranes in the oral cavities, thereby bypassing the hepatic clearance system (that is, the first pass metabolism).

Numerous oral film examples and formulations are known in the art. Specifically, oral thin fast dissolving films composed of water-soluble mucoadhesive polymers, which—when administered orally—dissolve rapidly in the saliva and allow the incorporated active ingredient(s) to be available for absorption through the oral mucosa. Oral thin fast dissolving films are therefore useful if the therapeutic objective is to achieve a rapid onset of action and to improve and maximize fast bioavailability of the drug by absorption through the oral mucosa and therefore avoiding the liver first pass metabolism, which the drug would otherwise be subjected to following ingestion. Certain actives present more of a challenge regarding effective absorption where, for example, such actives are complex molecules having low membrane permeability, and correspondingly low potential for bioavailability.

Thus, there exists a need for oral films with a controlled or sustained rate of dissolution employing disintegration, in which a slower release of active (drug) content from the polymer matrix is achieved. Such technology may have been reported for oral dosages in the form of tablets, but has not been effectively exploited for oral films. The transfer of technology from tablets to oral films is far from straightforward and involves a number of challenges, mostly related to the fabrication techniques. Challenges include the insolubility of certain biologically active ingredients, the need for taste-masking of bitter drugs, the development of an homogenous and stable viscous blend, finding a suitable drying time of film containing thermolabile drugs, ensuring that the product generate a strong and sustainable mucoadhesion, high dose incorporation in film, stability of film against humidity and temperature, the need for special packaging, and dose uniformity.

In the pharmaceutical industry the term disintegration is usually associated with the orally disintegrating tablet (ODT), which in general have low weight, small size, highly soluble components, and rapid disintegration. Such characteristics support the intended use of these products. Their disintegration times range from a few seconds to longer than a minute, but the large majority of these products have in-vitro disintegration times of approximately 30 seconds or less.

Thin film with non-self-aggregating uniform heterogeneity, process for their production and drug delivery systems made therefrom (CA2473967) focused on the fabrication of uniform films, but did not effectively describe a method for controlling the drug release or absorption profiles. Although the patent mentions the possible use of controlled release forms, it does not make any claims or explanations related to the strategies or mechanisms for an extended or fast drug release, or, mucosa or gastrointestinal drug site of absorption.

Disintegrable Oral Films (AU2007214474) describes slow disintegrating oral films containing a nicotine active for delivery and release into the oral cavity. It was disclosed a slow disintegrating oral film specific for maximizing the absorption of the nicotine active through the oral mucosa. The described technology is not necessarily applicable to other active ingredients. There remains a need for describing a strategy for fabricating oral films that are suitable for the slow release of a variety of active molecules. Moreover, the patent describes the use of specific film forming polymers, namely polyethylene oxide (100,000-8,000,000) and hydroxypropyl methylcellulose, or hydroxypropyl methylcellulose and xanthan gum, or hydroxypropyl methylcellulose and arabic gum. The patent did not provide an explanation or justification for the specific polymers, indicating that it may only be applicable to the nicotine oral film formulation, rather than a general formula for slow disintegrating oral films.

### SUMMARY OF THE DISCLOSURE

In one aspect of the present disclosure, it is provided an oral film formulation that disintegrates at a controlled or sustained rate followed or not followed by a dissolution at the end of the process, allowing a slower release of a given drug into the oral cavity.

In one aspect of the present disclosure there is provided an oral film formulation containing a desired level of a pharmaceutical or biological active component suspended/dissolved throughout a polymer matrix containing at least some inert hydrophobic material.

In one aspect of the disclosure, the disintegration mechanism is created by implementing sub-optimal conditions of pH or solubilizing power, which are not sufficient to ensure fast dissolution hence promoting disintegration.

In one aspect of the disclosure, the active agent is a complex molecule with low bioavailability when administered orally.

In one aspect of the disclosure, the oral film formulation employs an active agent that is a cannabinoid or derivative thereof.

In certain aspects of the disclosures, the polymeric matrix comprises non-swellable polymers or insoluble polymers which contribute at least 5% of the dry weight of the film.

In one aspect of the disclosure, the oral film formulation further comprises a sweetener.

In one aspect of the disclosure, the oral film formulation further comprises a flavor.

In one aspect of the disclosure, the oral film formulation further comprises a plasticizer.

In one aspect of the disclosure, the oral film formulation further comprises a surfactant.

In one aspect of the disclosure, the surfactant is Poloxamer.

In one aspect of the disclosure, the oral film formulation further comprises an emulsifier.

In one aspect of the disclosure, the oral film formulation further comprises the emulsifier is Ultralec P.

In one aspect of the disclosure, the oral film formulation is applied to the oral cavity of a mammal.

In one aspect of the disclosure, the oral film formulation adheres to the tongue or buccal cavity of the mammal.

In one aspect of the disclosure, the oral film formulation disintegrate within the mouth of a patient in less than about two minutes.

In one aspect of the disclosure, the oral film formulation disintegrates within the mouth of a patient in less than about five minutes.

In one aspect of the disclosure, the oral film formulation disintegrate within the mouth of a patient in more than about five minutes and less than about fifteen minutes.

In one aspect of the disclosure, it is disclosed a method of treating a medical condition comprising administering a pharmaceutical composition including a polymeric matrix at least partially composed of inert hydrophobic material, an effective amount of a pharmaceutically active component, and a permeation enhancer including a surfactant.

In another aspect of the disclosure, it is disclosed a method of treating a medical condition comprising administering an effective amount of a pharmaceutical composition, comprising a polymeric film matrix at least partially composed of inert hydrophobic material and a pharmaceutically active component including a cannabinoid in the polymeric matrix.

In one aspect of the present disclosure, there is provided an oral film formulation containing a desired level of a pharmaceutical or biological active component formed by the steps of, combining a drug, polymer, sweetener, flavor, plasticizer and/or surfactant; stirring them into water and other co-solvents until an homogeneous dispersion (wet blend) is obtained; coating the wet blend onto a support liner and drying it in an oven until the a suitable residual solvent level is reached; and cutting desired product to desired size, wherein polymers may be added gradually to wet blend, wherein additional drug solubilization techniques may be employed including complexation with cyclodextrins, reducing particle size, forming lipid dispersions, or creating intimate mixtures of drug and polymer in the solid state (amorphous solid dispersion); wherein the formulation may be further manipulated by adjusting solvent characteristics such as temperature and pH; wherein specific mixing manipulations may also be employed during the coating process, to ensure blend homogeneity and final product content uniformity and wherein non-swellable or insoluble polymer should contribute about 5 to 15% of the dry film weight, and the total polymer composition should be around 60% of the dry polymer weight.

In one aspect of the disclosure, the polymeric matrix is composed of up to 50% insoluble polymers selected from Ethyl Cellulose, polymethacrylate, polymethacrylate copolymer or a combination thereof.

In another aspect of the disclosure, the polymeric matrix is composed of up to 20% insoluble polymers selected from polyacrylic, poly(lactide-co-glycolide), polylactic acid, polyglycolic acid or a combination thereof.

In one aspect of the disclosure, the slow drug release from oral film formulation can also be applied in topical applications, to release small amounts of drug into the blood stream over a long period, particularly in transmucosal active agents, such as analgesic or antimicrobial agents in the wound care and drugs to treat angina and motion sickness.

In another aspect of the disclosure, the oral film formulation may be loaded with sensitive reagent to allow controlled release when exposed to a biological fluid or to create isolation barriers for separating multiple reagents to enable a timed reaction within a diagnostic device.

In one aspect of the disclosure, the oral film formulation contains a hydrophobic cannabinoid active ingredient. In this example, water-soluble polymers are used as the film formers. The formulation is based on two phases, an oil phase and an aqueous phase. The addition of an oil phase renders the film matrix an unfavorable environment for fast dissolution, thereby slowing down the release of drug from the polymer matrix.

In the embodiment above, poloxamer is used as a surfactant and Ultralec P is an emulsifier that promotes even blending and mixing. In the oil phase, the active (CBD) is first dissolved in a small amount of ethanol along with the alcohol-soluble flavor (L-Menthol). These are then dispersed in the carrier oil (medium chain triglycerides, MCT) along with other oil-soluble excipients, the antioxidant (α-tocopherol) and the flavor (peppermint oil). In the aqueous phase, the plasticizer (PEG), the polymer mixture consisting of hydroxypropyl cellulose (HPC), hydroxypropylmethyl cellulose (HPMC) and polyethylene oxide (PEO), the filler (talc), the sweetener (sucralose), and the preservative (methylparaben) are all combined in water.

These and other features, advantages and objects of the various embodiments will be better understood with reference to the following specification and claims.

### BRIEF DESCRIPTION OF THE DRAWINGS

FIG. 1 displays the difference between dissolution versus disintegration mechanisms, using CBD oral fil ms.

FIGS. 2A and 2B illustrate how the oral film formulation disintegrates slowly over time allowing a slower release of the drug into the aqueous media.

FIG. 3 illustrates the difference in drug release mechanisms between dissolving and disintegrating oral films.

FIG. 4 illustrates the Maropitant oral film disintegration mechanism in fake saliva: use of different surfactant in the formulation

FIG. 5 illustrates of Maropitant oral film disintegration versus dissolving film mechanisms in fake saliva: use of acid pH, neutral pH with maltitol, versus neutral pH without maltitol

FIG. 6 illustrates THC oral film disintegration mechanisms in fake saliva: use of sodium alginate.

### DETAILED DESCRIPTION OF THE DISCLOSURE

Film systems embody a field of technology that has major advantages in areas of administering various actives to an individual in need thereof. The present disclosure relates to oral films and methods for forming film products that include at least one active. Specifically, the disclosure provides for a film and a method of forming a film that controls the rate of release of certain molecules from the oral film matrix after oral administration.

Fast dissolving oral dosage forms may be effective in circumstances where the absorption of the drug through the oral mucosa is faster or equal to the dissolution of the drug in the saliva i.e. kabs≥kdiss. If, however, the absorption rate constant kabs of a given drug is lower than its dissolution rate constant kdiss in the saliva, kabs<kdiss, only a fraction will be absorbed transmucosally and a significant portion of the drug will be swallowed through the gastrointestinal track and subsequently metabolized in the liver. Such is the case for many active ingredients, for example, cannabidiol (CBD) or tetrahydrocannabinol (THC) that have very low oral bioavailability (only 5% when orally ingested), as they are first metabolized in the liver before entering the bloodstream. Additionally, both have a significantly delayed onset of their pharmacological action after ingestion (generally an average of over more than one hour after administration).

In order to address the problem of incomplete oral absorption of complex molecules like CBD or THC, it is critical to control the rate of release krel of these molecules from the oral film matrix after oral administration. If krel≤kdiss, the release of the drug from the film matrix becomes the rate controlling element for the absorption of the drug through the oral mucosa.

The solution disclosed herein is an oral film with a controlled or sustained rate of dissolution employing disintegration, in which a slower release of active (drug) content from the polymer matrix is achieved by using either erodible, water insoluble polymers or by adding components that limit the solubility of water-soluble polymers and make the environment unfavorable for polymer dissolution and drug release. This disintegration technology is applicable to a wide range of active ingredients but is especially useful for those that are rapidly soluble in saliva, or molecules with low membrane-permeability characteristics that would benefit from longer contact time with the mucosa.

The term “disintegrating” and variations thereof generally refers to the ability of the dosage forms to break up into small and submicron particles within a short period of time. The terms “disintegrating film”, “oral disintegrating film”, “disintegrable films” and “oral disintegrable films” refer to films in which the polymer matrix will break into smaller pieces that may or may not dissolve at a later stage, and will be referred to throughout as oral film(s) and oral film formulation(s).

Also used in the art to describe films include the terms “oral dissolving film”, “oral dissolvable film” “OSF”, “film”, “ODF”, “oral chewable film”, “OCF”, “oral thin film”, “OTF,” “oral drug strip” or “oral strip” which generally refer to films in which the polymer matrix will solubilize in the media.

ODF (Orally Disintegrating Film) and ODT (Orally Disintegrating Tablet) are two different dosage forms designed for oral administration. While both ODF and ODT share the characteristic of rapid disintegration in the mouth without the need for water, they differ in terms of their physical form and composition.ODF, or orally disintegrating film, is a thin, flexible, and typically transparent strip that dissolves quickly when placed on the tongue or in the buccal cavity. It is composed of a water-soluble or rapidly disintegrating polymer matrix that incorporates the active pharmaceutical ingredient (API) and other excipients. ODFs are designed to disintegrate or dissolve within seconds, delivering the drug for absorption through the oral mucosa or subsequent swallowing. On the other hand, ODT, or orally disintegrating tablet, is a solid dosage form that rapidly disintegrates in the mouth without the need for water. ODTs are compact tablets formulated with a combination of superdisintegrants, binders, and other excipients. The tablet structure allows for convenient handling and packaging. When placed on the tongue, an ODT rapidly disperses or disintegrates, forming a fine suspension or solution that can be swallowed without the need for chewing or water

The terms “blend” or “blending media” and variations thereof generally refers to the combination of the oral film formulation with the presence of solvents.

The term “drug absorption” or “absorption” as used in this specification, refers to the process of movement from the site of administration of a drug toward the systemic circulation, e.g., into the bloodstream of a subject.

The term “residence time” as used in the specification refers to the time taken by the film to disappear on the buccal mucosa.

Any number of active agents or active pharmaceutical ingredients may be included in the films discussed herein. The term “active(s)” or “active agent(s)” refers mainly to active pharmaceutical ingredients (APIs), but may also refer generally to any agent(s) that chemically interacts with the subject to which it is administered to cause a biological change, such as, but not limited to, eliminating symptoms of disease or regulating biological functions. The term “Pharmaceutical Ingredient or API” and variations thereof generally refers to any agent that is being administered orally to a subject and includes pharmaceutically active agents, nutraceutically active agents, and breath freshening agents.

Examples of pharmaceutically active agents include ACE-inhibitors, antianginal drugs, anti-arrhythmics, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies such as sildenafil citrate, tadalafil, and vardenafil, fertility agents, gastrointestinal agents, homeopathic remedies, hormones, hypercalcemia and hypocalcemia management agents, immunomodulators, immunosuppressives, anti-migraine preparations such as rizatriptan, eletriptan and zolmitriptan, motion sickness treatments, muscle relaxants, obesity management agents, osteoporosis preparations, oxytocics, parasympatholytics, parasympathomimetics, prostaglandins, psychotherapeutic agents, respiratory agents, sedatives such as lorazepam or diazepam, smoking cessation aids such as bromocryptine or nicotine, sympatholytics, tremor preparations, urinary tract agents, vasodilators, laxatives, antacids, ion exchange resins, anti-pyretics, appetite suppressants, expectorants, anti-anxiety agents such as alprazolam, anti-ulcer agents, anti-inflammatory substances, coronary dilators, cerebral dilators, peripheral vasodilators, psycho-tropics, stimulants, anti-hypertensive drugs, vasoconstrictors, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spadmodics, terine relaxants, anti-obesity drugs, erythropoietic drugs, anti-astmatics, cough suppressants, mucolytics, DNA and genetic modifying drugs, and combinations thereof. Examples of nutraceutically active agents include various dietary supplements, vitamins, minerals, herbs and nutrients.

The term “cannabis” is used to refer to plants of the genus Cannabis, including Cannabis sativa and Cannabis indica.

The term “cannabinoid” represents a group of C21 terpenophenolic compounds found uniquely in cannabis plants. Cannabinoids include the psychoactive compounds Δ9-tetrahydrocannabinol (THC), Δ8-THC, cannabinol (CBN), 11-hydroxy Δ9-THC, anandamide, and the non-psychoactive compounds cannabidiol (CBD), cannabichromene, and (-) Δ8-THC-11-oic acid. Cannabinoids can be synthetically made or can be extracted from the cannabis plant. The term cannabinoid is used herein to refer to a cannabinoid that is either synthetic or extracted from the plant. It is also used to refer to a single cannabinoid or mixture of cannabinoids.

“Bioavailability” as used herein in reference to active agents refers to the extent at which the active agent (drug or metabolite) enters systemic circulation, thereby accessing the site of action.

The term “polymer” refers to a long molecule chain made of many repeating units. The choice of polymers in an oral film formulation affect the mechanical and textural properties of the oral film formulation and drug release.

The term “matrix” or “film matrix” and variations thereof generally refers to the polymer component or mixture of polymers, “polymer matrix”, which creates the film forming matrix supporting the API within the oral film dosage form.

The term “water soluble polymers” and variations thereof generally refers to water soluble polymers that can be employed in the disclosed films and include water soluble cellulose derivatives, including hydroxypropylmethyl cellulose, hydroxyethyl cellulose, hydroxypropyl cellulose; polyvinyl pyrrolidone (PVP); copovidone (a copolymer of 1-vinyl-2-pyrrolidone and vinyl acetate); other copolymers of vinyl pyrrolidone; other polymers or copolymers of substituted vinyl pyrrolidone; derivatives of polyvinyl pyrrolidone; polyethylene oxide, carboxymethyl cellulose; polyvinyl alcohol; natural gums, including xanthan, tragacanth, guar, acacia and arabic gums; and water soluble polyacrylates. Combinations of these water-soluble polymers or other water-soluble polymers can also be used. Examples of substituted vinyl pyrrolidones include but are not limited to N-vinyl-3-methyl-2-pyrrolidone, N-vinyl-4-methyl-2-pyrrolidone, N-vinyl-5-methyl-2-pyrrolidone, N-vinyl-5,5-dimethyl-2-pyrrolidone, N-vinyl-3,3,5-trimethyl-2-pyrrolidone and others. Examples of monomers that can be copolymerized with vinyl pyrrolidone or substituted vinyl pyrrolidones include vinyl aromatic monomers such as styrene, and acrylate or methacrylate monomers such as methyl methacrylate and 2-dimethylaminoethyl methacrylate. A water-soluble polymer is a polymer that can be diluted in water, with or without the assistance of co-solvents and other neutralizing agents, to form transparent homogenous solutions. Water-soluble polymers may be synthetic such as polyethylene oxides (PEOs), polyvinyl pyrrolidones (PVPs), polyvinyl alcohol (PVOH, PVA), or may be naturally occurring such as pullulan, sodium alginate, xanthan gum, carrageenan, gelatin, guar gum, and gum Arabic.

Water-insoluble polymers include cellulose, ethyl cellulose (EC), poly(lactide-co-glycolide) (PLGA) or polylactic acid or poly glycolic acid, and polyvinyl acetate-based polymers. The buccal or sublingual film dosage form can comprise a single film layer, or multiple layers. In some embodiments, a bilayer or multilayer film would include a mucoadhesive layer containing the API which is placed against the oral mucosa and a second layer directed outwards from the mucosa serving as a protective barrier against abrasion from the tongue or mastication. This protective layer also serves to favor the directed absorption of the API within the oral cavity rather than enteric uptake in the gastrointestinal (GI) tract.

The term “mucoadhesive” or “bioadhesive” means that the composition of the film layer is formulated to adhere to the mucous membrane through which delivery of the active agent is targeted. For example, bioadhesive polymers used in formulating the film should be selected to exhibit adequate adhesion within the environment at the targeted mucous membrane to ensure that the bioadhesive layer remains in contact with the mucous membrane to which it is applied and allows the active agent to directly enter the blood stream through the mucous membrane.

The term “mucoadhesive film former” refers to polymers that form the film matrix, film strip, film sheet and dissolves in aqueous environment and give bio-adhesive properties to the mucosa, having examples comprising PEO, Pullulan, CMC, HPC, HPMC and exclude ethyl cellulose (EC), polyvinyl alcohol (PVA), Starch and Polymethacrylate polymers. Examples of mucoadhesive materials that can be used to prepare the mucoadhesive particles include poly(ethylene oxide), polyvinyl pyrrolidone, poly(acrylic acid) derivatives (e.g., commercially available Carbopol®), polycarbophil, polyoxyalkylene ethers, polymethacrylates, polymethacrylates-based copolymers (e.g., commercially available Eudragit®), biodegradable polymers such as poly(D,L-lactide-co-glycolide) (e.g., commercially available Resomer®), anionic biopolymers such as hyaluronic acid, or sodium carboxymethylcellulose, cationic biopolymers such as chitosan or poly(L-lysine) and other cellulose derivatives. Other mucoadhesive polymers that can be used include methyl vinyl ether-maleic acid, a mixed salt of sodium/calcium methyl vinyl ether-maleic acid, methyl vinyl ether-maleic anhydride, and half esters (monoethyl; monobutyl and isopropyl ester) of methyl vinyl ether-maleic anhydride copolymers (e.g., commercially available Gantrez®).

The term “instantly wettable” and variations thereof generally refers to the ability of the film dosage form to rapidly imbibe moisture upon oral administration to a subject and immediately soften, whereby the subject is prevented from experiencing a prolonged adverse feeling in the mouth, and with respect to certain aspects of the disclosure refers to embodiments in which moisture (i.e., water) applied to a surface of the film penetrates the thickness of the film (e.g., typically about 5 μm to 200 μm) within 10, 15 or 20 seconds. The wettability also ensures quick mucoadhesion ensuring the film sticks to the mucosa and stays in place.

The term “suspending agent” (also referred to as a “viscosity increasing agent”) refers to water soluble ingredients or non-water soluble ingredients or combination thereof employed to prevent adjacent suspended particles from coming close enough to join each other by increase sufficiently the viscosity of the drug vehicle, and enables by steric stabilization the suspension to be stably maintained, beside above properties certain suspending agent/viscosity increasing agent additionally interact with biological mucosa to create and strengthen oral film mucoadhesion. Examples comprise polysaccharide in the form of one or a mix of Hydroxypropylmethylcellulose (HPMC) where the polymer structure combines both hydrophobic (methoxy group) and hydrophilic substitutions (hydroxypropoxy group) where the 2% aqueous viscosity is between about 1298 to about 5040 millipascal second (mPas) (2%, Hydroxypropyl Cellulose (HPC) where the 2% aqueous viscosity is above about 150 mPas (2%, 25C), hydroxyethyl cellulose (HEC), Gums such as water soluble carboxymethyl cellulose (CMC), Gellan, propylene glycol alginate, water soluble alginate salt, Acacia, Pectin, Xanthan, guar gum, carrageenan, and water insoluble alginates derivatives, water insoluble CMC derivatives, colloidal silicon dioxide, Agar, Locust bean, tragacanth. It may also comprise Polyvinylpyrrolidone of Molecular Weight (MW) of 1 000 000 MW and above (K-value of 85 and above) with aqueous viscosity of 300 mPAs (10%, 20C) and above and higher molecular weight polyethylene oxide (PEO) (MW above 600 000). The following are excluded from the definition of the terms “suspending agent/viscosity increasing agent”: one or a mix of HPMC where the polymer structure do not combines both hydrophobic and hydrophilic substitutions, and or having aqueous viscosity below 1298 mPas or above 5040 mPas (2%, 20C), Methyl cellulose (MC), Microcrystalline cellulose (MCC), powdered cellulose, Sodium Starch Glycolate, starch, Polyvinylpyrrolidone of MW below 1.000.000 MW and K-value below 85 and with aqueous viscosity of less than 300 mPAs (10%, 20C), polyvinylpyrrolidone-vinyl acetate copolymer, polyplasdone crospovidone, HPC where the 2% aqueous viscosity is below 150 mPas (2%, 25C), water insoluble bentonite.

The term “surfactant” refers to excipients that are employed to dissipate the free surface energy of particles by reducing the interfacial tension and contact angle between the solid and the suspending vehicle, and comprise PEG 300 oleic glycerides (Labrafil® M-1944CS), PEG 300 linoleic glycerides (Labrafil® m-2125C5); Hydroxylated lecithin; Caprylocaproyl polyoxyl-8 glycerides; Polyoxyethylene (4) sorbitan monostearate, Polyoxyethylene 20 sorbitan tristearate, Polyoxyethylene (5) sorbitan monooleate, Polyoxyethylene 20 sorbitan trioleate; Sorbitan Esters (Sorbitan Fatty Acid Esters) such as: Sorbitan monolaurate, Polyoxyethylene Sorbitan Fatty Acid Esters such as: Polyoxyethylene 20 sorbitan monolaurate, Polyoxyethylene (4) sorbitan monolaurate, Polyoxyethylene 20 sorbitan monopalmitate, Polyoxyethylene 20 sorbitan monostearate, Polyoxyethylene 20 sorbitan monooleate, Polyoxyethylene sorbitan monoisostearate Polyethylene glycol monostearate (Gelucire 48/16), poloxamer having MW up to 14.600, viscosity up to 3100 mPAs (77C) but exclude surfactant(s) of an HLB below 7 such as Propylene glycol monocaprylate type I, Propylene glycol monocaprylate type II, Propylene glycol monolaurate, Sorbitan monoisostearate, Sorbitan monooleate, Sorbitan monopalmitate, Sorbitan monostearate, Sorbitan sesquioleate, Sorbitan trioleate, Sorbitan tristearate and/or glyceryl monoleate.

The term “therapeutically effective amount” refers to an amount of a pharmaceutically active agent, which when administered to a particular subject, considering the subject's age, weight and other relevant characteristics, will attenuate, ameliorate, or eliminate one or more symptoms of a disease or condition that is treatable with the pharmaceutically active agent.

The term “flavoring agent” or flavor” and variations thereof generally refers to concentrated preparations, with or without flavor adjuncts required in their manufacture, used to impart flavor, with the exception of salt, sweet, or acid tastes. Flavoring agents may be classified as natural, artificial, or natural and artificial (N&A) by combining the all natural and synthetic flavors or other forms known in the art. Flavouring agents are categorized by their physical classification as solid flavoring agents and liquid flavoring.

The term “flavor enhancer” and variations thereof generally refers to compounds that particularly enhance certain tastes or reduce undesirable flavors without having an especially strong taste of their own. Flavor enhancers harmonize taste components and make food and drug preparations more palatable. Examples include but are not limited to maltol, ethyl maltol and monosodium glutamate, glutamic acid, glutamates, purine-5-ribonucleotides, inosine, guanosine, adenosine 5-monophosphates, sugars, sweetener, carboxylic acids (e.g., citric, malic, and tartaric), common salt (NaCl), amino acids, some amino acid derivatives (e.g., monosodium glutamate—MSG), and spices (e.g., peppers) are most often employed, yeast, yeast extract, dried yeast and others or mixtures thereof.

The term “sweetener” and variations thereof generally refers to a solid or liquid ingredient that is used to impart a sweet taste to food or drug product. Sweeteners are often classified as either nutritive (caloric) or non-nutritive (non-caloric), natural or synthetic. Examples of sweeteners include but are not limited to sucrose, dextrose, lactose, glucose, advantame, sorbitol, mannitol, liquid glucose, honey molasses, saccharin, sucralose, rebaudioside A stevia, rebaudioside M stevia, stevioside, mogroside IV, mogroside V, alitame, saccharin, neohesperidin dihydrochalcone, cyclamate, neotame, N-[3_(3-hydroxy-4-methoxybenzyl yl) propyl]-L-α-aspartyl]-L-phenylalanine 1-methyl ester, N-[3-(3-hydroxy-4-methoxyphenyl)-3-methylbutan yl]-L-α-aspartyl]-L-phenylalanine 1-methyl ester, N-[3-(3-methoxy-4-hydroxyphenyl) propyl]-L-α-aspartyl]-L-phenylalanine 1-methyl ester, curculin, cyclamate, aspartame, acesulfame potassium and others or mixtures thereof.

The term “taste maskers”, in the context of pharmaceuticals and formulations, are substances or compounds used to minimize or disguise the unpleasant taste or bitterness of active pharmaceutical ingredients (APIs) or other ingredients in oral dosage forms. The taste of certain drugs or ingredients can be unpalatable, which can negatively impact patient compliance and acceptance of the medication. Taste maskers work by interfering with the taste perception of the bitter or unpleasant components, either by physically blocking taste receptors or by chemically interacting with taste receptors on the tongue. They can enhance the overall flavor profile of the formulation, making it more palatable and improving patient acceptance. Taste maskers can include a variety of substances such as sweeteners, flavors, bitter blockers, and taste modifiers. Sweeteners, such as sucrose or artificial sweeteners, help to counterbalance bitterness and improve the taste. Flavors, such as fruit flavors or mint flavors, add pleasant sensory experiences to mask the undesirable taste. Bitter blockers can selectively inhibit bitter taste receptors, reducing the perception of bitterness. Taste modifiers can alter the perception of taste by interacting with taste receptors or modifying the chemical properties of the compound

The term “plasticizer” refers to a component that reduces the glass-transition temperature of the film forming polymers (e.g., the water soluble polymer or water soluble polymers in the film). The plasticizer increases the flexibility, enhances elasticity and reduces brittleness of the film. Examples of plasticizers include triacetin, triethyl citrate, tributyl citrate, acetyl tributyl citrate, acetyl triethyl citrate, trioctyl citrate, acetyl trioctyl citrate, trihexyl citrate, dibutyl sebacate, PEG 300, PEG 400 and Glycerine, etc.

The term “treating”, “treat” or “treatment” as used herein embraces both preventative, i.e., prophylactic, and palliative treatment, i.e., relieve, alleviate, or slow the progression of the patient's disease, disorder or condition. The term “treat” or “treatment” in the context of a mental health disorder such as addiction, depression, anxiety and posttraumatic stress disorder (PTSD) refers to any treatment of a disorder or disease associated with a mental health disorder, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treat” is used synonymously with the term “prevent”.

Hydrophobic excipients are composed of non-polar molecules insoluble in water and soluble in non-polar solvents.

The term “unfavorable environment” for polymer dissolution refers to an environment that does not enable the polymer matrix to dissolve freely. This limited dissolution can be due to insufficient solubility or pH environment that promotes a slower disintegration rather than a dissolution.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or method steps.

As above, a variety of additives that can be integrated into the films may provide a variety of different functions. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active ingredient(s).

### Disintegrating Versus Dissolving Films

The present disclosure describes oral disintegrating film formulations that provide different delivery characteristics compared to conventional oral fast dissolving films. Instead of quickly dissolving in the oral cavity, disintegrating formulations break down at a controlled rate followed or not by a dissolution at the end of the process. This will favor a slower release of the drug into the oral cavity thereby avoiding saturation of the oral mucosal membranes and increasing mucosal absorption.

As aforementioned, oral fast-dissolving film formulations are composed of water-soluble polymers, which rapidly dissolve in the saliva upon oral administration. Water-soluble polymers form molecular dispersions in water and any dispersed drugs will immediately dissolve. Therefore, by using water-soluble polymers, the fast dissolving films formulation technology ensures that the drug is available for rapid absorption through the oral mucosa.

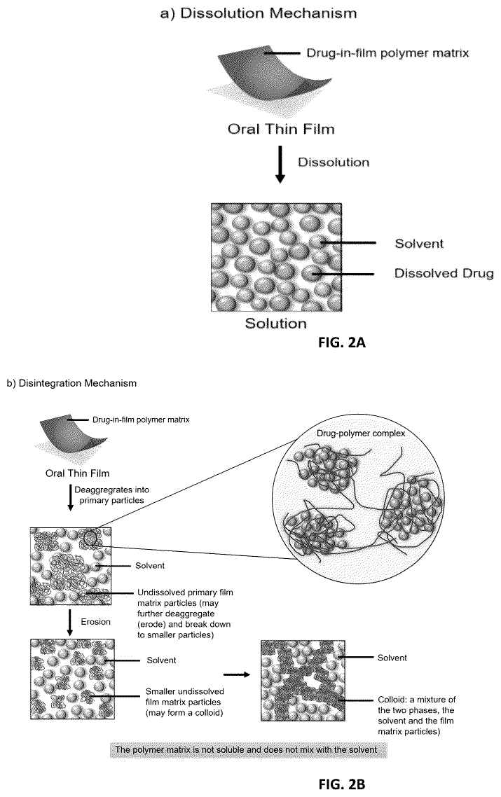

On the other hand, oral films that are based on disintegrating formulation technology consist of a mixture of soluble and insoluble polymers that are insoluble or practically insoluble in water or soluble polymer in an unfavorable environment but disintegrate upon contact with aqueous media like saliva. Such disintegrating polymers de-agglomerate into subunits after they are exposed to an aqueous medium. The active drug remains entrapped or in intimate contact within these polymer matrix sub-units and will only be released and become available for solubilization in the saliva upon further erosion or slow solubilization of these sub-units. Hence, the proposed disintegrating film technology employs the disintegration and erosion rate of the polymer to control the rate at which the drug dissolves in the saliva. FIGS. 2A and 2B illustrate the difference in drug release mechanisms between dissolving and disintegrating oral films.

According to embodiments, it is disclosed an oral film formulation that employs the disintegration and erosion rate of polymers to control drug release from the oral film matrix into the oral saliva, thereby allowing for increased buccal/oral mucosal absorption, extended time of action and reduced adverse effects and increased bioavailability by avoiding first-pass metabolization. This technology is applicable to a wide range of active ingredients but is especially useful for those that are rapidly soluble in saliva, or molecules with low membrane-permeability characteristics.

Largely, the release kinetics of drugs from the polymer matrix are primarily dependent on the physicochemical properties of the materials used as well as the morphology of the system. With regard to disintegrating films, the release of the drug is markedly influenced by the polymer disintegration rate and erosion of the film, although variation in pH or temperature and the types of plasticizer or surfactant may cause increase or decrease in the erosion or disintegration rates of polymers. In the case of soluble drugs, upon contact with biological fluids, the polymeric film starts to swell following polymer chain relaxes, resulting in drug diffusion. The release of the drug holds a direct relationship with polymer structure; for example, linear amorphous polymers dissolve much faster than cross-linked or partially crystalline polymers.

Depending on the properties of the film matrix material, film matrix systems are defined as either hydrophilic or hydrophobic. In hydrophilic film matrix systems, the drug is dispersed throughout a polymer matrix of hydrophilic material. The rate of drug release is controlled by both diffusion and erosion. When water is absorbed by the matrix, the film matrix swells and the surface polymer hydrates. The polymer changes from a solid or crystalline state to a gel state, forming a gel layer on the surface that controls the rate of release of the drug. As the gel layer increases, the polymer chains closest to the surface begin to relax and lose consistency, which is followed by a gradual erosion of the matrix. Water-soluble drugs dissolve and are released by a combination of diffusion out of the film matrix depending on drug water solubility, through the gel layer, and as a result of the erosion of the film matrix itself. Many factors affect the rate of drug release from hydrophilic film matrix systems, including the concentration of polymer in the matrix, particle size of the polymer, the viscosity of the polymer in solution, and solubility of the drug itself.

In hydrophobic film matrix systems, the drug is dispersed throughout a polymer matrix of inert hydrophobic material. The hydrophobic film matrix undergoes no or minimal swelling on contact with water. When water enters the matrix, the drug dissolves and is predominately released by diffusion out of the film matrix depending on its water solubility. In such diffusion-based film matrix systems, the drug is not uniformly released over time, because the diffusion front of the drug gradually moves further into the matrix. In addition, because drug solubility is usually dependent on pH, the rate of release of the drug varies with the pH of the environment. Consequently, the rate of drug release from hydrophobic film matrix systems is altered by factors such as non-uniform loading of the drug within the matrix, and the incorporation of pH modifiers.

### Polymeric Excipients Used in Oral Films

The polymeric excipients used in oral films can be subdivided into groups according to their solubility and swelling characteristics. Examples are listed in Table 1.

TABLE 1
Examples of Common Polymers used
in Oral Film Matrix Preparation.

| Polymer properties | | Examples |
|---|---|---|
| Soluble/Erodible | Swellable | Hydroxypropylmethyl cellulose |
| | | (HPMC) |
| | | Sodium carboxymethyl cellulose |
| | | (CMC) |
| | | Polyethylene Oxide (PEO) |
| | | Pullulan, sodium alginate, |
| | | xanthan gum, carrageenan, |
| | | gelatin, guar gum and gum Arabic |
| | Non-swellable | Polyvinyl pyrrolidones (PVPs) |
| | | Hydroxypropyl cellulose (HPC) |
| Insoluble | Non-swellable | Polyvinyl acetate |
| | | Polyester |
| | Swellable | Eudragit ® RS (polymethacrylates) |
| | | Sodium carboxymethyl cellulose |
| | | (CMC) |
| | | Ethyl Cellulose |
| | | Polyacrylic |
| | | poly(lactide-co-glycolide) |
| | | polylactic acid |
| | | poly glycolic acid |

A water-soluble polymer is a polymer that can be diluted in water, with or without the assistance of co-solvents and other neutralizing agents, to form transparent homogenous solutions. Water-soluble polymers may be synthetic such as polyethylene oxides (PEOs), polyvinyl pyrrolidones (PVPs), polyvinyl alcohol (PVOH, PVA), or may be naturally occurring such as pullulan, sodium alginate, xanthan gum, carrageenan, gelatin, guar gum, and gum Arabic. Water-insoluble polymers include cellulose, ethyl cellulose (EC), acrylic polymers, and polyvinyl acetate-based polymers.

The solubility of polymers can be tailored synthetically by setting a covalent or hydrogen-bonding crosslinking point. For example, cellulose is insoluble in water; however, cellulose derivative: hydroxypropyl methyl cellulose (HPMC), carboxymethyl cellulose (CMC) and methyl cellulose (MC) are characterized by the pronounced affinity of their chemical structures for aqueous solutions in which they swell rather than dissolve. Their solubility and swellability is also influence by the molecular mass of the polymer.

As mentioned earlier, the dissolution medium (ie, water or saliva) penetration into the oral film matrix is controlled by the nature of the film polymeric carrier. This is dependent on many factors, including: polymer molecular weight, content, and substitution type; Interactions of the polymer mixture with the surrounding environment (dissolution medium, the drug itself and other excipients)

The swellable soluble/erodible polymers hydrate instead of disintegrating when in contact with water. Entry of the solvent hydrates and swells the polymer, consequently, relaxes the polymer chains, and decreases the glass transition temperature (Tg) forming a gel. Soluble drugs are released via diffusion through the gel layer, whereas insoluble drugs, are liberated via erosion of the surrounding film matrix structure. On the other hand, for non-swellable soluble/erodible and insoluble polymers, the drug particles are solely released by erosion of the film surface into smaller subunits over time. The drug can only diffuse into the medium once it is released from the polymer network. This will also be the case of soluble polymers that are present in an environment that does not favor their dissolution. Interactions of a soluble polymer mixture with the surrounding environment (dissolution medium, the drug itself and other excipients) may lead to disfavor their dissolution, thereby slowing down the drug release (see Table 2). In some embodiments, the drug is Maropitant.

In some embodiments, non-swellable or insoluble polymers should contribute at least 5 to 15% of the dry film weight, and the total polymer composition should be around 60% of the dry polymer weight.

Manufacturing an oral film involves combining the film ingredients (ie, drug, polymer, sweetener, flavor, plasticizer, surfactants, etc) and stirring them into water and other co-solvents until a homogeneous dispersion (wet blend) is obtained. The wet blend is then coated onto a support liner and dried in an oven until a suitable moisture level is reached. The obtained product is cut to a desired size and packaged.

During the blending stage, conventional mixing techniques are employed for dissolving or dispersing the polymer into the wet blend composition. Polymers are usually added gradually to the wet blend and mixed until completely dissolved or dispersed. Depending on the polymer nature and desired film characteristic, additional polymer solubilization techniques may be employed. Solubility enhancing techniques include reducing particle size, forming lipid dispersions, or creating intimate mixtures of drug and polymer in the solid state (amorphous solid dispersion). Moreover, solubility can be manipulated by adjusting characteristics such as temperature and pH. For example, pectin and starch are only soluble at high temperatures, chitosan is only soluble in acidic solutions and sodium carboxymethyl cellulose is more soluble in neutral and basic pH.

Depending on the blend physical nature, specific mixing manipulations may also be necessary during the coating process, to ensure blend homogeneity and final product content uniformity. Coating or casting methods are particularly useful for forming oral films as disclosed herein. Specific examples include reverse roll coating, forward roll coating, gap or knife over roll coating, air knife coating, curtain coating, or combinations thereof, especially when a multi-layered film is desired.

The oral film formulation of the present disclosure may be formed into a sheet prior to drying. After the desired components are combined to form a multi-component matrix, including the polymer, water/organic solvent/hydro-alcoholic solutions, active other components as desired, the combination is formed into a sheet or film, by any method known in the art such as, coating, spreading, casting or drawing the multi-component matrix. A multi-layered film may be achieved by coating, spreading, or casting a combination onto an already formed film layer. Although a variety of different film-forming techniques may be used, it is desirable to select a method that will provide a flexible oral film, such as reverse roll coating. The flexibility of the oral film allows for the sheets of oral film to be rolled and transported for storage or prior to being cut into individual dosage forms. Desirably, the oral film will also be self-supporting or in other words able to maintain their integrity and structure in the absence of a separate support. Furthermore, the films of the present invention may use selected materials that are edible or ingestible.

Because the taste of certain actives may be unpleasant, it is often beneficial to add a sweetener, flavoring agent, refreshing agent, taste-masking agent, or a combination of these materials. According to certain embodiments, examples of sweeteners that can be used in the disclosed film dosage forms include acesulfame potassium, aspartame, aspartan-acesulfame salt, cyclamate, erythritol, glycerol, glycyrrhizin, hydrogenated starch hydrolysate, isomalt, lactitol, maltitol, mannitol, neotame, polydextrose, saccharin, sorbitol, sucralose, tagatose, xylitol, dextrose, glucose, fructose, and honey. Flavoring agents that can be added to the disclosed film dosage forms include isoamyl acetate (banana flavor), benzaldehyde (cherry flavor), cinnamaldehyde (cinnamon flavor), ethyl propionate (fruit flavor), methyl anthranilate (grape flavor), limonene (orange flavor), ethyl decadienoate (pear flavor), allyl hexanoate (pineapple flavor), ethyl meltol, ethylanillin (vanilla flavor), and methyl salicylate (wintergreen flavor). Refreshing agents, also called cooling agents, are chemicals that trigger the cold sensitive receptors creating a cold sensation. Refreshing agents that can be added to the oral dosage forms disclosed herein include menthol, thymol, camphor and eucalyptol.

Plasticizers can be advantageously employed in the film formulations as needed to suitably modify the flexibility of the film to facilitate processing and allow the film to easily conform to the shape of the oral mucosa to which the film is applied. Plasticizers that can be effectively employed in the disclosed film oral dosage forms to improve flexibility of the film can be selected from ethylene glycol, propylene glycol, tributyl citrate, triethyl citrate, glycerol, and combinations of two or more thereof. Depending on the selected film-forming polymers and other components of the film formulation, a suitable amount of plasticizer is typically from about 0.1% to 10%, 0.5% to 5%, or 1% to 5% dry weight of the film.

Bulking agents or fillers may be added as desired to increase the size of the finished film product to facilitate processing and manufacturing, or to modify properties (e.g., increase or decrease residence time or increase stiffness) of the film formulation. Suitable fillers that can be added to the disclosed film products include starch, calcium salts, such as calcium carbonate, and sugars, such as lactose. The amount of fillers that can be added to the film oral dosage forms disclosed herein are typically up to about 25%, to 20%, 1% to 15% or about 2% to about 10% of the weight of the film on a dry basis.

As disclosed herein, the slow drug release from oral disintegrating films can be useful for developing controlled or sustained-release formulations. These have several advantages of over fast dissolving formulation, which result in immediate drug release. The advantages include improved patient compliance due to less frequent drug administration, reduction of fluctuation in steady-state drug levels, maximum utilization of the drug, increased safety margin of potent drug, reduction in healthcare costs through improved therapy and shorter treatment period.

The slow drug release from oral disintegrating films can also be applied in topical applications, to release small amounts of drug into the blood stream over a long period. This may be useful for delivery of transmucosal active agents, such as analgesic, anti fungal, and antimicrobial.

Oral disintegrating films may be loaded with sensitive reagent to allow controlled release when exposed to a biological fluid or to create isolation barriers for separating multiple reagents to enable a timed reaction within a diagnostic device.

### Exemplary Embodiments

The following is an exemplary embodiment of an oral film formulation containing a hydrophobic cannabinoid active ingredient. Cannabidiol (CBD) or tetrahydrocannabinol (THC) both have a very low oral bioavailability (only 5% when orally ingested), as they are first metabolized in the liver before entering the bloodstream. Additionally, both have a significantly delayed onset of their pharmacological action after ingestion (generally an average of over an hour after administration).

In this embodiment, water-soluble polymers are used as the film formers. The formulation is based on two phases, an oil phase and an aqueous phase. The addition of an oil phase renders the film matrix an unfavorable environment for fast dissolution, thereby slowing down the release of drug from the polymer matrix.

To homogenously incorporate the hydrophobic active into the oil-in-water emulsion formulation, a mixture of surfactants and emulsifiers are used. Specifically, poloxamer is a hydrophilic non-ionic surfactant composed of a triblock copolymer with a central hydrophobic block of polypropylene glycol flanked by two hydrophilic blocks of polyethylene glycol (PEG). Additionally, Ultralec P is an emulsifier that promotes even blending and mixing.

In the oil phase, the active (CBD) is first dissolved in a small amount of ethanol along with the alcohol-soluble flavor (L-Menthol). Next, a dispersion is formed in the carrier oil (medium chain triglycerides, MCT) along with other oil-soluble excipients, the antioxidant (α-tocopherol) and the flavor (peppermint oil).

In the aqueous phase, the plasticizer (PEG), the polymer mixture consisting of hydroxypropyl cellulose (HPC), hydroxypropylmethyl cellulose (HPMC) and polyethylene oxide (PEO), the filler (talc), the sweetener (sucralose), and the preservative (methylparaben) are all combined in water.

After mixing the oil and water phases, the wet blend is cast over liner rolls then subject to drying to form a film sheet.

The formulation is illustrated in Table 2.

TABLE 2
Example of a Disintegrating Film for a Hydrophobic Cannabinoid
Active Ingredient

| | | | % dry | w/film | % wet | Weight |
|---|---|---|---|---|---|---|
| Phases | Order | Excipients | (w/w) | (mg) | (w/w) | (g) |
| Oil | 5 | MCT oil | 2.85 | 2.12 | 0.76 | 0.28 |
| | 1 | CBD | 13.44 | 10.00 | 3.59 | 1.32 |
| | 2 | Ethanol | x | x | 1.20 | 0.44 |
| | 3 | L-Menthol | 2.55 | 1.89 | 0.68 | 0.25 |
| | 4 | α-Tocopherol | 0.31 | 0.23 | 0.08 | 0.03 |
| | 6 | Peppermint oil | 9.06 | 6.74 | 2.42 | 0.89 |
| Aqueous | 2 | Water | x | x | 72.13 | 26.55 |
| | 1 | PEG | 5.40 | 4.02 | 1.44 | 0.53 |
| | 3 | Poloxamer | 5.40 | 4.02 | 1.44 | 0.53 |
| | 4 | Methylparaben | 0.51 | 0.38 | 0.14 | 0.05 |
| | 5 | Sucralose | 2.75 | 2.05 | 0.73 | 0.27 |
| | 6 | Talc | 5.40 | 4.02 | 1.44 | 0.53 |
| | 7 | HPC-medium | 20.16 | 15.00 | 5.38 | 1.98 |
| | | viscosity | | | | |
| | 8 | HPMC low | 21.59 | 16.06 | 5.76 | 2.12 |
| | | viscosity | | | | |
| | 8 | HPMC medium | 2.75 | 2.05 | 0.73 | 0.27 |
| | | viscosity | | | | |
| | 8 | PEO medium | 6.72 | 5.00 | 1.79 | 0.66 |
| | | viscosity | | | | |
| | 9 | Ultralec P | 1.12 | 0.83 | 0.30 | 0.11 |
| | | Total Wet: | x | x | 100.00 | 36.81 |
| | | Total Dry: | 100.00 | 74.39 | 26.68 | 9.82 |

In the disclosed exemplary formulation, the polymer mixture (HPC, HPMC and PEO), in this specific oil-based environment, get their rate of solubilization in water reduced but disintegrate upon contact with aqueous media like saliva. Once they encounter an aqueous medium, the polymer mixture will de-agglomerate into subunits, but the CBD remains entrapped in these polymer matrix sub-units and will only be released and become available for solubilization in the saliva upon further erosion of these sub-units. Therefore, this oral film formulation helps maintain an acceptable CBD concentration in the oral cavity solution for a longer time, thereby increasing the likelihood for its buccal/mucosal absorption.

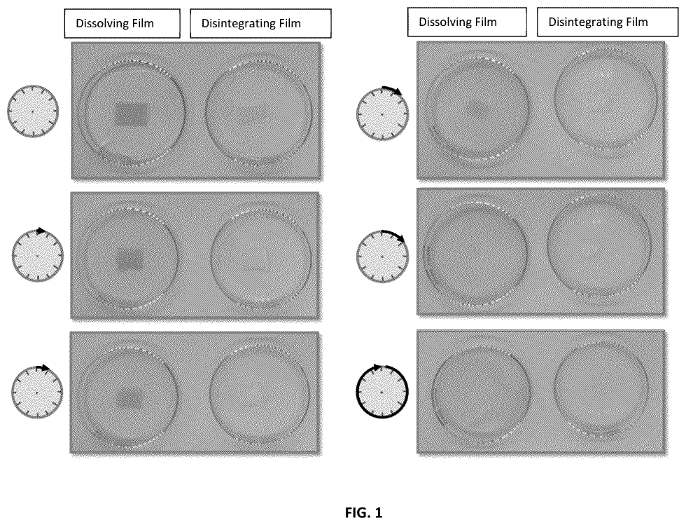

FIG. 1 displays the drug release mechanisms of two CBD oral films, one developed based on dissolving polymer technology dissolves and quickly releases CBD into solution, whereas the other developed based on the disintegrating polymer technology (formulation given in Table) slowly disintegrates and controllably releases CBD into solution.

Disclosed disintegration test employs the use of 40 ml fake saliva filled in petri dish, dispense a representative size of the ODF on the surface of the test solution, with camera record the disintegration or dissolution sequence. Said fake saliva 1 L preparation includes dissolving in 1 L of purified USP water the following:
2.38 g Na2HPO4 (Sodium phosphate Dibasic),
0.19 g KH2PO4 (Potassium phosphate Monobasic)
8.00 g NaCl (Sodium Chloride)
Adjust the final pH to pH 6.75 with phosphoric acid

Table 3 provides an example of an oral film formulation in which water-insoluble polymers (ethyl cellulose and Eudragit® RL (polymethacrylates) are used as the film formers.

TABLE 3
Example of a Disintegrating Film fabricated
with insoluble polymers

| | | Amount | % wet | % dry | |
|---|---|---|---|---|---|
| Ingredients | function | (g) | (w/w) | (w/w) | mg/film |
| Ethanol | solvent | 10 | 36.51 | — | |
| Water | solvent | 10 | 36.51 | — | |
| THC | Active | 1 | 3.651 | 13.532 | 10.00 |
| PEG | plasticizer | 1.2 | 4.381 | 16.238 | 12.00 |
| Alpha tocopherol | stabilizer | 0.01 | 0.037 | 0.135 | 0.10 |
| Propylen paraben | anti-bacterial | 0.01 | 0.037 | 0.135 | 0.10 |
| Menthol | flavor | 0.15 | 0.548 | 2.03 | 1.50 |
| Sucralose | sweetner | 0.15 | 0.548 | 2.03 | 1.50 |
| HPC | film former | 1.17 | 7.272 | 15.832 | 11.70 |
| PVP | film former | 3 | 10.953 | 40.595 | 30.00 |
| Tween 80 | surfactant | 0.5 | 1.825 | 6.766 | 5.00 |
| HPMC | film former | 0.2 | 0.73 | 2.706 | 2.00 |
| Total wet | | 27.39 | | | |
| Total dry | | 7.39 | 100 | 100 | 73.90 |

In the above example, the film former polymers are dissolved in a suitable solvent (ethanol) to form a homogenous blend with the remaining excipients. Since the polymer mixture is insoluble in water, once the dry film encounters an aqueous medium, the polymer mixture will not dissolve but rather de-agglomerate into subunits, and the API (CBD) will remain entrapped in these polymer matrix sub-units and will only be released and become available for dissolution in the saliva upon further erosion of these insoluble polymer sub-units.

In this example, an oral film formulation contains a hydrophobic active ingredient (maropitant) as outlined below. In order to homogenously incorporate the hydrophobic active ingredient into the water, a mixture of Tween, a polysorbate-type nonionic surfactant, and hydroxylated lecithin, an emulsifier that promotes even blending and mixing, are used. In the aqueous phase, the plasticizer (glycerin), Maropitant, surfactants (Tween, hydroxylated lecithin) are well homogenized then the polymers mixture (HPC, HPMC and PEO) are combined. After mixing, the wet blend is cast over liner rolls then subject to drying to form a film sheet. The formulation details are outlined in Table 4.

TABLE 4
Example of a Disintegrating Film for a Hydrophobic
Active Ingredient, Maropitant

| | | Weight | % Wet | % Dry | Weight (mg) |
|---|---|---|---|---|---|
| Order | Excipients | (g) | (wt/wt) | (wt/wt) | per Film |
| 5 | Maropitant | 1.5 | 8.48 | 31.95 | 60.00 |
| 4 | Hydroxylated Lecithin | 0.4 | 2.26 | 8.52 | 16.00 |
| 2 | Polysorbate 80 | 0.1 | 0.57 | 2.13 | 4.00 |
| 3 | Water | 13 | 73.47 | — | — |
| 1 | Glycerin | 0.235 | 1.33 | 5.01 | 9.40 |
| 7 | HPMC high viscosity | 0.1 | 0.57 | 2.13 | 4.00 |
| 8 | HPC low viscosity | 0.5 | 2.83 | 10.65 | 20.00 |
| 10 | HPMC medium | 0.16 | 0.90 | 3.41 | 6.40 |
| | viscosity | | | | |
| 11 | PEO medium viscosity | 1.7 | 9.61 | 36.21 | 68.00 |

| | | | | |
|---|---|---|---|---|
| TOTAL | 17.70 | 100.0 | 100.0 | 187.80 |

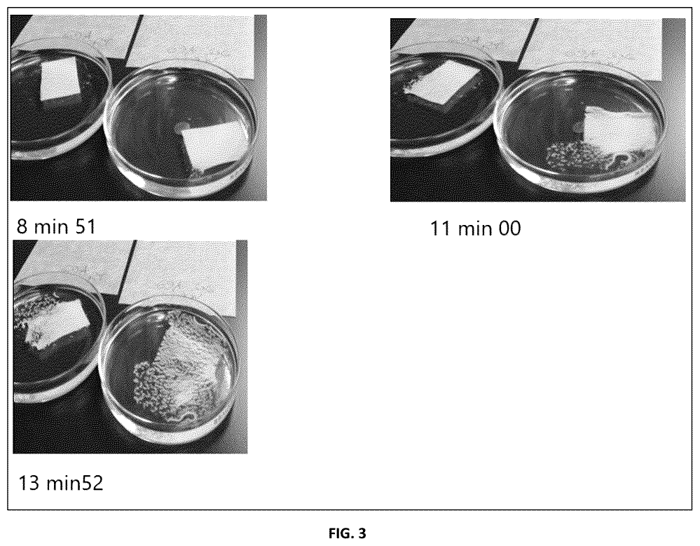

The oral film formulation disintegrates quickly and further erosion occurs slowly over time allowing a slow release of the drug into the aqueous media. Although this formulation is a good example of disintegrating film technology, its disintegration properties are specifically tailored to the active therein, however, those formulations would equally be applicable to similar active and actives in the same classes and from the same family. As seen in FIG. 3.

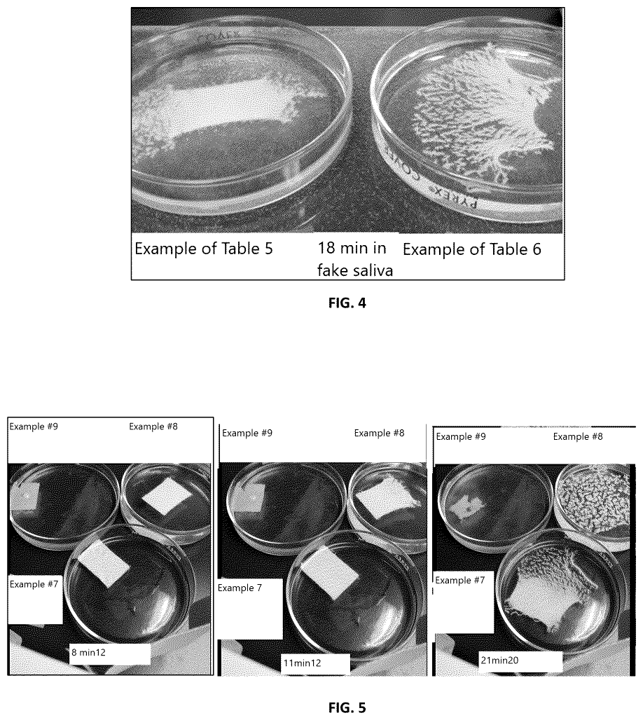

FIG. 4 displays the drug release mechanisms of Maropitant films developed based on disintegrating polymer technology (formulations with different surfactants are given in Table and Table 6.)

TABLE 5
Example of a Disintegrating Film for a Hydrophobic
Active Ingredient, Maropitant

| Order | Excipients | weight | % wet | % dry | w/film |
|---|---|---|---|---|---|
| 4 | Maropitant | 3.00 | 8.43 | 31.28 | 60.00 |
| 3 | Hydroxylated | 1.00 | 2.81 | 10.43 | 20.00 |
| | Lecithin | | | | |
| 2 | water | 26.00 | 73.05 | — | — |
| 1 | Polyethylene | 0.47 | 1.32 | 4.90 | 9.40 |
| | glycol | | | | |
| 5 | HPMC high | 0.20 | 0.56 | 2.09 | 4.00 |
| | viscosity | | | | |
| 6 | HPC low | 1.00 | 2.81 | 10.43 | 20.00 |
| | viscosity | | | | |
| 7 | HPMC | 0.32 | 0.90 | 3.34 | 6.40 |
| | medium | | | | |
| | viscosity | | | | |
| 8 | PEO medium | 3.34 | 9.38 | 34.83 | 66.80 |
| | viscosity | | | | |
| 8 | PEO medium | 0.26 | 0.73 | 2.71 | 5.20 |
| | viscosity | | | | |
| | total wet | 35.59 | 100.00 | — | — |
| | total dry | 9.59 | — | 100.00 | 191.80 |

TABLE 6
Example of a Disintegrating Film for a Hydrophobic
Active Ingredient, Maropitant

| Order | Excipients | weight | % wet | % dry | w/film |
|---|---|---|---|---|---|
| 4 | Maropitant | 3.00 | 8.43 | 31.28 | 60.00 |
| 3 | Labrafil | 1.00 | 2.81 | 10.43 | 20.00 |
| 2 | water | 26.00 | 73.05 | — | — |
| 1 | Polyethylene | 0.47 | 1.32 | 4.90 | 9.40 |
| | glycol | | | | |
| 5 | HPMC high | 0.20 | 0.56 | 2.09 | 4.00 |
| | viscosity | | | | |
| 6 | HPC low | 1.00 | 2.81 | 10.43 | 20.00 |
| | viscosity | | | | |
| 7 | HPMC | 0.32 | 0.90 | 3.34 | 6.40 |
| | medium | | | | |
| | viscosity | | | | |
| 8 | PEO medium | 3.34 | 9.38 | 34.83 | 66.80 |
| | viscosity | | | | |
| 8 | PEO medium | 0.26 | 0.73 | 2.71 | 5.20 |
| | viscosity | | | | |
| | total wet | 35.59 | 100.00 | — | — |
| | total dry | 9.59 | — | 100.00 | 191.80 |

FIG. 5 displays the drug release mechanisms of Maropitant films developed based on disintegrating polymer technology where the ODF has basic pH of 8 (Table 7) and in another example with maltitol a pore former used to accelerate the disintegration (Table 8) versus dissolving polymer technology where the citric acid creates acidic pH favorable for Maropitant solubilization (Table 9.)

It is was observed that the acidic pH of the film is the main driving condition for disintegration of Maropitant film.

TABLE 7
Example of a Disintegrating Film for a Hydrophobic
Active Ingredient, Maropitant: ODF pH 8.0

| Order | Excipients | weight g | % wet | % dry | w/film |
|---|---|---|---|---|---|
| 5 | API | 1.5 | 8.48 | 31.95 | 60.00 |
| 4 | Hydroxylated | 0.4 | 2.26 | 8.52 | 16.00 |
| | Lecithin | | | | |
| 2 | tween 20 | 0.1 | 0.57 | 2.13 | 4.00 |
| 3 | water | 13 | 73.47 | — | — |
| 1 | Glycerin | 0.235 | 1.33 | 5.01 | 9.40 |
| 6 | HPMC E4M | 0.1 | 0.57 | 2.13 | 4.00 |
| 7 | HPC ssl | 0.5 | 2.83 | 10.65 | 20.00 |
| 8 | HPMC E50 | 0.16 | 0.90 | 3.41 | 6.40 |
| 9 | PEO 200K | 1.7 | 9.61 | 36.21 | 68.00 |
| | tot wet | 17.70 | 100.00 | — | — |
| | tot dry | 4.70 | — | 100.00 | 187.80 |

TABLE 8
Example of a Disintegrating Film for a Hydrophobic Active
Ingredient, Maropitant: ODF pH 8.0 with Maltitol

| Order | Excipients | weight g | % wet | % dry | w/film |
|---|---|---|---|---|---|
| 5 | API | 1.5 | 8.30 | 29.62 | 60.00 |
| 4 | Hydroxylated | 0.4 | 2.21 | 7.90 | 16.00 |
| | Lecithin | | | | |
| 3 | tween 20 | 0.1 | 0.55 | 1.97 | 4.00 |
| 2 | water | 13 | 71.96 | — | — |
| 1 | Glycerin | 0.235 | 1.30 | 4.64 | 9.40 |
| 6 | HPMC E4M | 0.1 | 0.55 | 1.97 | 4.00 |
| 7 | HPC ssl | 0.5 | 2.77 | 9.87 | 20.00 |
| 8 | Maltitol | 0.37 | 2.05 | 7.31 | 14.80 |
| 9 | HPMC E50 | 0.16 | 0.89 | 3.16 | 6.40 |
| 10 | PEO 200K | 1.7 | 9.41 | 33.56 | 68.00 |
| | tot wet | 17.70 | 100.00 | — | — |
| | tot dry | 4.70 | — | 100.00 | 187.80 |

TABLE 9
Example of a Dissolving Film for a Hydrophobic Active
Ingredient, Maropitant: ODF pH 4.91 with Citric acid

| Order | Excipients | weight g | % wet | % dry | w/film |
|---|---|---|---|---|---|
| 5 | API | 1.5 | 8.30 | 29.62 | 60.00 |
| 4 | Hydroxylated | 0.4 | 2.21 | 7.90 | 16.00 |
| | Lecithin | | | | |
| 2 | tween 20 | 0.1 | 0.55 | 1.97 | 4.00 |
| 6 | Citric acid | 0.37 | 2.05 | 7.31 | 14.80 |
| 3 | water | 13 | 71.96 | — | — |
| 1 | Glycerin | 0.235 | 1.30 | 4.64 | 9.40 |
| 7 | HPMC E4M | 0.1 | 0.55 | 1.97 | 4.00 |
| 8 | HPC ssl | 0.5 | 2.77 | 9.87 | 20.00 |
| 9 | HPMC E50 | 0.16 | 0.89 | 3.16 | 6.40 |
| 10 | PEO 200K | 1.7 | 9.41 | 33.56 | 68.00 |
| | tot wet | 18.07 | 100.00 | — | — |
| | tot dry | 5.07 | | 100.00 | 202.60 |

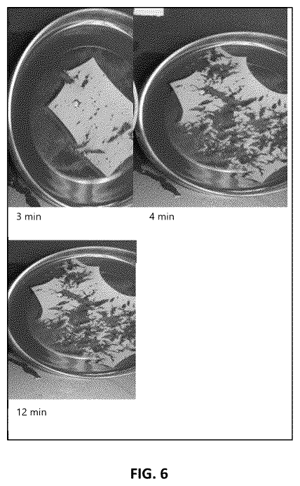

FIG. 6 displays the drug release mechanisms of THC films developed based on disintegrating polymer technology where the ODF contain sodium alginate. In the disclosed exemplary formulation (Table 10), the polymer mixture (sodium alginate, HPMC and PEO), in this specific oil-based environment, get their rate of solubilization in water reduced but disintegrate upon contact with aqueous media like saliva. The disintegration characteristic are tested using a limited amount of liquid in a petri dish without agitation.

TABLE 10
Example of a Disintegrating Film for
a Hydrophobic Active Ingredient, THC

| | % wet | % dry |
|---|---|---|
| Ingredients | (w/w) | (w/w) |
| Purified water | 76.142 | — |
| USP | | |
| Tween 80 | 3.045 | 12.766 |
| PEG | 2.538 | 10.638 |
| Glycerol | 0.888 | 3.723 |
| Menthol | 0.152 | 0.638 |
| THC isolate | 2.538 | 10.638 |
| Alpha tocopherol | 0.05 | 0.212 |
| Propylen paraben | 0.025 | 0.106 |
| Menthol | 0.457 | 1.915 |
| Sucralose | 0.330 | 1.383 |
| Maltitol | 0.431 | 1.809 |
| PEO | 10.152 | 42.553 |
| HPMC | 2.538 | 10.638 |
| Sodium Alginate | 0.711 | 2.979 |
| Total wet | 100.000 | — |
| Total dry | — | 100.000 |

In FIG. 2A, dissolution is the process of dissolving a substance in a solvent, thereby forming a solution. The components of a solution are mainly of two types, solutes and the solvent. When dissolving in solvent, the solute disperses/dissociates to form molecular level, chemically and physically homogeneous dispersion, called solution. In FIG. 2B, disintegration, on the other hand, is the physical process that occurs when a dosage form (in this case the oral film) breaks up into smaller particles. The foregoing usually occurs in two steps; the dosage form initially breaks up into primary particles that further disaggregate. Therefore, an orally disintegrating technology is intended to help maintain an acceptable drug concentration in the oral cavity solution for a longer time, thereby increasing its buccal/mucosal absorption. This process also implies that mucoadhesion is retained at all times to ensure the product is not swallowed.

The above description is considered that of the preferred embodiment(s) only. Modifications of these embodiments will occur by those skilled in the art and by those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

1. An oral film formulation comprising:
a) at least one hydrophobic active agent;
b) a polymeric matrix at least partly composed of hydrophobic or insoluble excipients, or a combination thereof, to facilitate a controlled or sustained dissolution;
wherein the oral film formulation has an unfavorable environment for fast dissolution, exhibits partial disintegration within 1 to 10 minutes and a sustained rate of dissolution as confirmed by a disintegration test conducted in a limited volume petri dish, and wherein the film undergoes disintegration into smaller and smaller pieces.

2. The oral film formulation of claim 1, further comprising an emulsifier selected from the group of glycerol monooleate, Glyceryl monolinoleate, polysorbate, SLS, carboxymethyl cellulose, guar gum, lecithin, carrageenan, acacia gum, or a combination thereof.

3. The oral film formulation of claim 1, further comprising a surfactant selected from the group of Sorbitan monolaurate, polysorbate, cremophor, poloxamer, labrafil, labasol, transcutrol, sodium lauryl sulfate, or a combination thereof.

4. The oral film formulation of claim 1, wherein the active agent is a cannabinoid or derivative thereof.

5. The oral film formulation of claim 1, wherein the polymeric matrix makes up about 50% of the dry weight of the film.

6. The oral film formulation of claim 1 wherein the polymeric matrix is composed of up to 50% insoluble polymers which are ethyl cellulose, polymethacrylate, a polymethacrylate copolymer or a combination thereof.

7. The oral film formulation of claim 1, wherein the polymeric matrix is composed of up to 50% soluble polymers which are hydroxypropyl methylcellulose, polyethylene glycol, hydroxypropyl cellulose, sodium alginate or a combination thereof.

8. The oral film formulation of claim 1, wherein the polymeric matrix is composed of up to 20% insoluble polymers which are polyacrylic, poly (lactide-co-glycolide), polylactic acid, poly glycolic acid or a combination thereof.

9. The oral film formulation of claim 1, further comprising an antioxidant selected from the group of cysteine, sodium metabi-sulfite (SMB), propyl gallate (PG), butylated hydroxytoluene (BHT), butylated hydroxyanisole, alpha tocopherol (vitamin E), ascorbic acid, ascorbyl palmitate, citric acid, phosphoric acid, sodium sulfite, tocopheryl polyethylene glycol succinate (TPGS), or a combination thereof.

10. The oral film formulation of claim 1, further comprising one or more taste maskers selected from the group consisting of sweeteners, flavors, bitter blockers, and taste modifiers.

11. An oral film formulation comprising:
a) Maropitant as the active agent;
b) a polymeric matrix composed of about 50% soluble polymers selected from the group consisting of Hydroxypropyl Methylcellulose, Polyethylene Oxide, Hydroxypropyl Cellulose, or a combination thereof;
c) a surfactant; and/or an emulsifier
d) a plasticizer, and
e) a pH rage from 6 to 9 promoting an unfavorable environment for dissolution
wherein the oral film formulation has an unfavorable environment for fast dissolution, exhibits slow disintegration and gradual erosion, enabling a controlled and sustained release of the drug into the aqueous media.

12. An oral film formulation according to claim 11, further comprising a pore former.

13. The oral film formulation of claim 12, wherein the pore former is maltitol.

14. An oral film formulation of claim 11, wherein the formulation is intended for animal use.

15. An oral film formulation of claim 11, wherein the formulation is specifically designed for the treatment of animals for anti-emesis.

16. An oral film formulation comprising:
a) a cannabinoid active agent
b) a water-soluble polymer mixture;
c) an oil phase rendering the environment unfavorable for fast dissolution; thereby slowing the release of the cannabinoid active agent from the polymer mixture;
d) and
e) a surfactant; and/or an emulsifier;
wherein, upon encountering an aqueous medium, the polymer mixture deagglomerates into subunits, and wherein the cannabinoid active agent remains entrapped within the polymer mixture subunits until further eroded, released and solubilized in saliva.

17. The oral film formulation of claim 16, wherein the water-soluble polymer mixture comprises hydroxypropyl cellulose (HPC), hydroxypropylmethyl cellulose (HPMC) and polyethylene oxide (PEO).

18. The oral film formulation according to claim 1, comprising a second active agent.

19. The oral film formulation according to claim 1, where disintegration initially starts within 1 to 10 minutes and is visually completed in not less than 4 minutes.

20. An oral film formulation according to claim 1, further comprising an additional excipient selected from the group consisting of stabilizers, pH modifiers, and taste maskers.
